(19) United States
(12) Patent Application Publication
(10) Pub. No.: US 2012/0123277 A1
(43) Pub. Date: May 17, 2012

(54) METHOD FOR DETECTING AN EXTRAORDINARY SITUATION

(75) Inventors: Armin Blaha, Wien (AT); Emanuel Preuschl, Wien (AT); Werner Kurschl, Linz (AT); Christofer David, Klosterneuberg (AT); Michael Affenzeller, Linz (AT)

(73) Assignee: SPANTEC GMBH, Wien (AT)

(21) Appl. No.: 13/254,665

(22) PCT Filed: Feb. 23, 2010
(86) PCT No.: PCT/AT10/00047

§ 371 Date: Feb. 3, 2012

(30) Foreign Application Priority Data
Mar. 2, 2009 (AT) A 340/2009

(51) Int. Cl. A61B 5/103; A61B 6/00; A61B 5/11

(52) U.S. Cl. 600/476; 600/587; 600/595

(57) ABSTRACT

A method for detecting an abnormal situation of a person, a contact measuring device which the person wears directly or indirectly on the body and which is a contact sensor and/or a pressure sensor and/or a photosensor and/or a distance measuring device being used to determine a contact state and/or the pressure exerted by a body part, such as a foot of a person, onto a reference surface, such as a floor, so that an abnormal situation can be detected among a plurality of situations.

The present inventions relates to a method for detecting an abnormal situation of a person.

EP 1278457B1, for example, describes a method for detecting a situation which is abnormal for a person, especially the fall of a person. The method disclosed in this document is based on the detection of an abnormal situation of a person based on measured values related to bodily functions, such as heart rate, a characteristic blood pressure value, respiratory rhythm and/or respiratory rate. A similar method is also described in Kang et al., Monitoring of Activities of Daily Living in Home Environment, Proceedings of 1^{st} Joint International Pre-Olympic Conference of Sport Science & Sport Engineering, Volume I: Computer Science in Sports, p 97-102.

The document EP 1974662A1 discloses a method which detects an abnormal situation of a person based on the change of the kinetic energy of a person, which is measured based on the acceleration of a person, over a period of time.

EP 0849715A2 discusses a method which detects the fall of a person based on a change in the inclination and position of the person as well as on measured values of the velocity and acceleration sequences of a person.

In practice, the data are often interpreted incorrectly, which especially leads to false alarms due to alleged falls of a person, so that the above-mentioned methods are not efficient.

The aim of the present invention consists in detecting a situation which is abnormal for a person based on a very simple measurement, said simple measurement being controlled by further, more complicated measurements. According to the invention, this aim is achieved by detecting a contact status and/or a pressure exerted, for example, by the foot of a person on a reference surface, such as the floor, by means of a contact measuring device, such as a contact sensor and/or a pressure sensor and/or a photosensor, and/or a distance measuring device which the person wears directly or indirectly on the body, so that an abnormal situation is detectable among various conditions based on a change of the contact status and/or the pressure exerted by a body part, such as the foot of a person, on a reference surface, such as a floor.

The person wears the contact measuring device, such as a contact sensor or a pressure sensor and/or a photosensor, and/or the distance measuring device directly or indirectly on the body. In the framework of the disclosure of the present invention, the sensors may be attached to the body by means of adhesive tape or patch-like applications. The sensors may be worn indirectly, if they are, for example, integrated into a piece of clothing and/or an accessory item which the person wears, such as into the sole of a shoe.

Within the framework of the present invention, it is not excluded that the method discussed herein may also be applied in the same way for detecting an abnormal situation of an animal, such as a horse.

In the framework of the present invention, a situation which is abnormal for a person refers, for example, to the fall of an elderly person. In normal situations, assuming that an elderly person will not run, an elderly person will always have at least one foot on the ground. Even if the person runs, the period of time during which the running person does not contact the floor directly or indirectly with none of their two feet, never amounts to more than 500 ms. If one of the person's feet does not exert any pressure onto the floor and if it is still not possible to detect a contact or pressure status after 1,000 ms, based on these measured values, the detected event is to be considered as an abnormal situation, such as a fall.

By means of the contact measuring device, such as a contact sensor or a pressure sensor and/or a photosensor, and the distance measuring device which is integrated into a shoe, this condition may be monitored easily and efficiently. A normal condition may be detected based on the measurement of values, such as the sequence of steps, etc.

In case of a fall, both feet of the elderly person will no longer contact the floor.

At the same time, the methods described herein may also be used for detecting the movements of athletes, such as runners, in order to examine the motion sequence for abnormal situations, such as injuries or incorrect movements.

The same holds true for the analysis of the sequence of motions of a dressage horse during a competition or during training.

The advantage of measuring the contact or pressure state as a primary measurement signal is that a simple signal which can be detected in a highly efficient way constitutes a good basis for further differentiations which are not obligatory and may be based on further measurements.

It is not always possible to clearly interpret an abnormal situation which is detected by means of the contact measuring device merely based on the contact or pressure state. An elderly person whose feet, according to the measurements, do not contact the floor may have fallen, but could also sit on a chair, keeping the feet off the floor. In order to be able to detect a situation in which the person may be harmed (fall), it is reasonable to apply further algorithms in order to be able to further differentiate the situations.

The invention further comprises the determination of the person's physiological index which is then related to the contact state and/or the pressure.

The physiological index refers to the determination of at least one measured physiological value, such as heart rate and body conductivity (perspiration), over a certain period of time. The duration of this period is determined depending on the values to be detected.

The measured physiological values before, during and after an event which is harmful for a person are substantially different from the values measured during normal events.

The method for detecting abnormal situations can be extended by determining an activity index of the person which is then related to the contact state and/or the pressure and/or the physiological index.

The activity index refers to the determination of a value in relation to the kinetic energy of the body over a certain period of time. Again, the duration of this period is determined depending on the values to be detected.

The values in relation to the kinetic energy of a person's body before, during and after an event which is harmful for a person are substantially different from the values measured during normal events.

The method according to the invention may comprise the output of a signal when an abnormal situation is detected and/or to be expected.

Such a signal may be transmitted as an information to the person who is in an abnormal situation and subsequently or simultaneously to third persons. Advantageously, there is a device emitting the signal attached to the person in the abnormal situation and at other locations.

Signals may be generated in the form of siren alarms, sounds, stimuli, messages via telephone or other media as well as in all forms which are known to those skilled in the art.

The above-described devices for detecting an abnormal situation are characterized by the fact that an abnormal situation can be predicted on the basis of the collected values, even before it has occurred, with a high likelihood of the predictions being correct. A feeling of vertigo (physiological value) or a person stumbling (detection of the sequence of steps by determining the contact state) before a fall constitute examples of values which are used for such predictions. It is part of the method of the invention that a signal is transmitted, before the harmful situation occurs.

The method of the invention can be extended by making it possible for the person affected or another person to control the signal output. It has proved advantageous for the output of signals to be controlled by the person affected or a third person, before or when an abnormal situation occurs, in order to avoid false alarms.

According to the invention, it is possible to determine the physiological index based on measured physiological values, such as heart rate, blood pressure, skin conductivity (perspiration), body temperature, etc, these values being preferably averaged over a certain period of time and/or evaluated by means of an analysis method.

In the framework of the present invention, it is also possible to determine the physiological index by comparing measured physiological values, such as pulse, blood pressure, skin conductivity (perspiration), body temperature, etc, of one part of the body to the measured values, such as pulse, blood pressure, skin conductivity (perspiration), body temperature, etc, of another part of the body, said values being preferably averaged over a certain period of time and/or evaluated by an analysis method.

The method of the invention may also include the determination of the activity index of the person based on activity values, such as acceleration, movements, position, etc, said values being preferably averaged over a certain period of time and/or evaluated by an analysis method.

The method of the invention may be complemented by comparing the activity values, such as acceleration, movements, position, etc, of one part of the body of the person to the activity values, such as acceleration, movements, position, etc, of another part of the body of the person, said values being preferably averaged over a certain period of time and/or evaluated by an analysis method.

The values for the determination of the physiological index and the activity index are measured at suitable locations on the person's body. Body conductivity and perspiration, for example, can be easily and efficiently measured in the area behind a person's ears. Carrying out measurements in this area has the advantage that the devices required for the measurement may be integrated into a hearing aid device. The same holds true for carrying out measurements in other locations where the person wears some aid devices; it is, thus, possible to integrate the measurement of the heart rate into a watch, for example.

The measured values may be analyzed according to one or a combination of the following methods or according to a method which is known to those skilled in the art, for example by means of a threshold analysis or a Fourier analysis.

The time interval between the individual measurements is preferably selected in order to make sure that the measurement data can be analyzed in the best possible way and that the amount of data can be efficiently processed. The above-mentioned analysis methods are preferably complemented by self-learning algorithms.

The measurement of values at or relating to a first body part and further body parts constitutes a further possibility of differentiating detected conditions. For the interpretation of measurement results, it may be important to measure the position of one part of the body in relation to other parts of the body. The same holds true for the measurement of physiological values.

A device for implementing the method according to the invention comprises a contact measuring device, such as a contact sensor and/or a pressure sensor and/or a photosensor, and/or a distance measuring device for determining the contact state or the pressure exerted by a body part on a reference surface; said device may further comprise one of the following devices:
measuring device for determining the physiological index,
measuring device for determining the activity index,
device for generating signals,
device for evaluating data,
position measuring device.

A device for implementing the method according to the invention may preferably comprise data evaluation devices in addition to the above-mentioned measuring devices, said data evaluation devices being used for evaluating the state of health and the behavior of the person in everyday life in addition to the detection of abnormal situations of the person. The data evaluation can be carried out for shorter periods of time, for evaluating a person's daily routine, for example, or for longer periods of time, for evaluating the recovery progress after an injury or surgery, for example.

Moreover, the device may also comprise devices for determining the position of the person. Locating the position or transmitting the position of the person to other persons or other devices may be restricted to cases of abnormal situations of the person.

A device for implementing the method of the invention can be designed in a way that it consists of a first unit and optionally a second unit, the first unit comprising at least one of the devices and the second unit optionally comprising the same or at least one other device, the first unit and the second unit communicating with each other.

A device for implementing the detection of an abnormal situation of a person according to the invention may also comprise devices, said devices comprising a device for monitoring a person's position, so that a signal is generated if the device or a unit of the device are removed from the person and/or the communication between the first unit and the second unit is interrupted.

Especially elderly people often forget to take the device with them, to attach the device on their bodies, or they remove it, because it is temporarily uncomfortable. Athletes can easily lose the device when exercising. The above-described generation of a signal is triggered in all of these and similar cases.

A device for implementing the method of the invention can be designed in a way that the device or at least one unit of a device which is attached to the person is integrated.

In the framework of the present invention it is possible, as mentioned above, to integrate the device or at least individual units for carrying out the detection into medical aid devices, such as hearing aids, glasses, heart rate measuring devices. This significantly improves the comfort for the person wearing the device, as the person is used to wearing those medical aid devices.

It is also possible, within the framework of the present invention, to integrate the device or individual units into pieces of clothing or accessories, such as watches, jewelry, etc.

The device for implementing the method of the invention may be configured in a way that to and/or further measured values will only be captured and/or transmitted from the first unit to the second unit, if, based on at least one measured value, the occurrence of an abnormal situation is considered to be possible and/or established. Creating a communication link between the first unit and further units mainly serves energy saving purposes in order to supply energy to the mobile units worn by the person for as long a period of time as possible.

The above-described set-up of the method, the way in which a value which is easy to measure is used as a first evidence for the detection of an abnormal situation, and the further interpretation of the first measured value based on measured values concerning the physiological index and the activity index allow for establishing a communication link between the individual units.

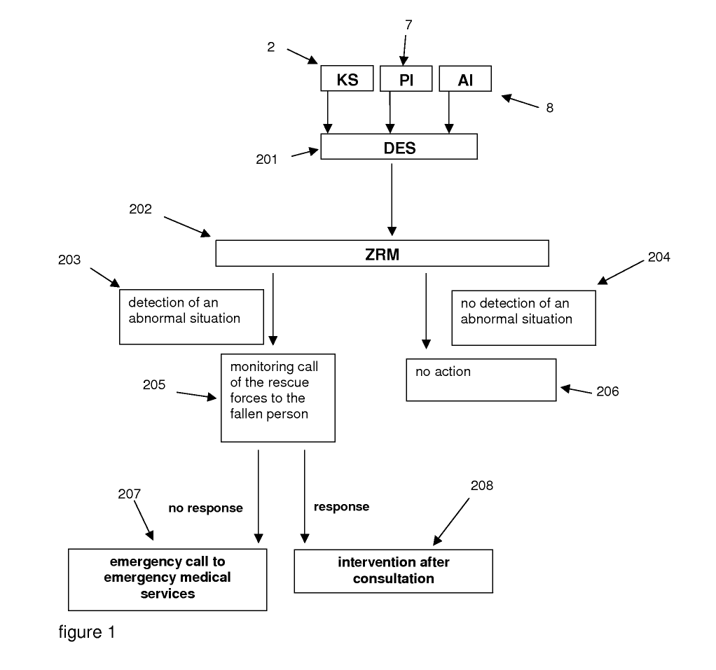

FIG. 1 shows a flow diagram for detecting whether a person is in an abnormal situation.

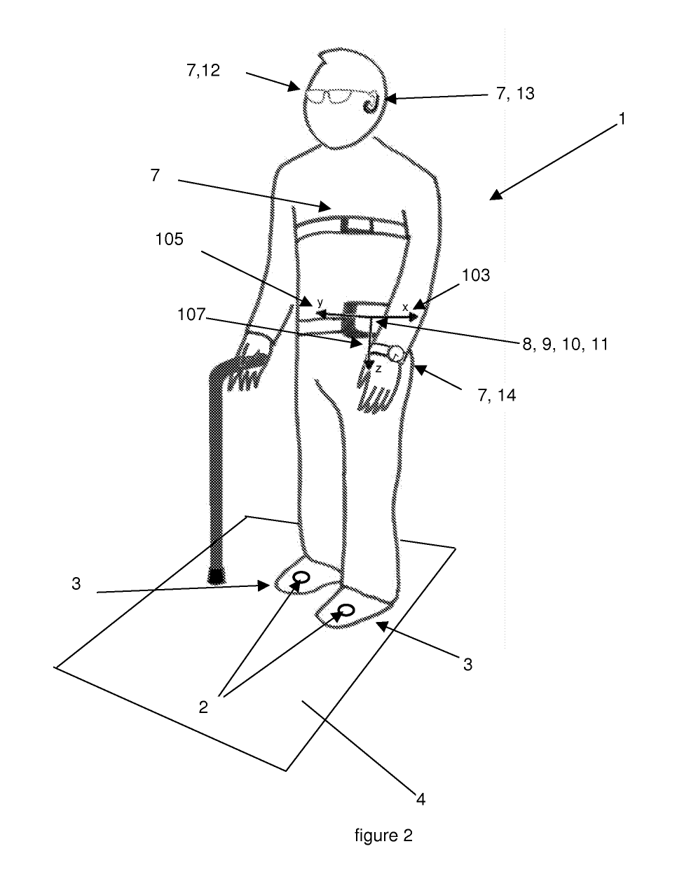

FIG. 2 shows an exemplary set-up of the measuring device necessary for the implementation of the method of the invention.

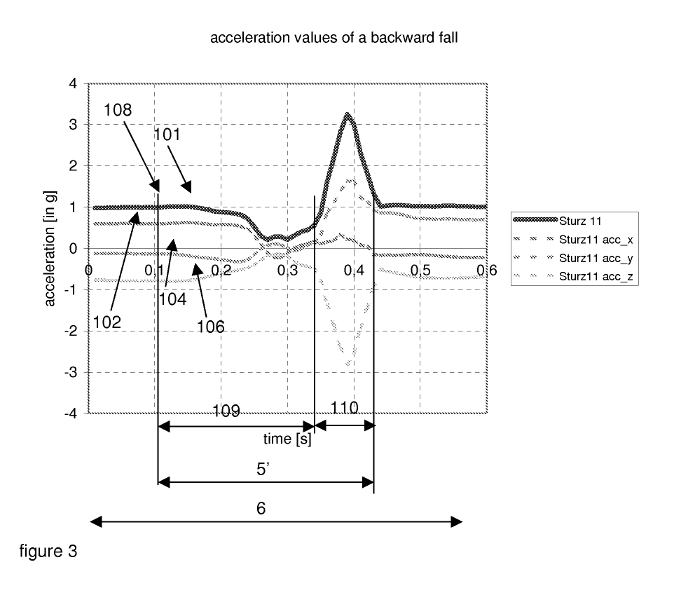

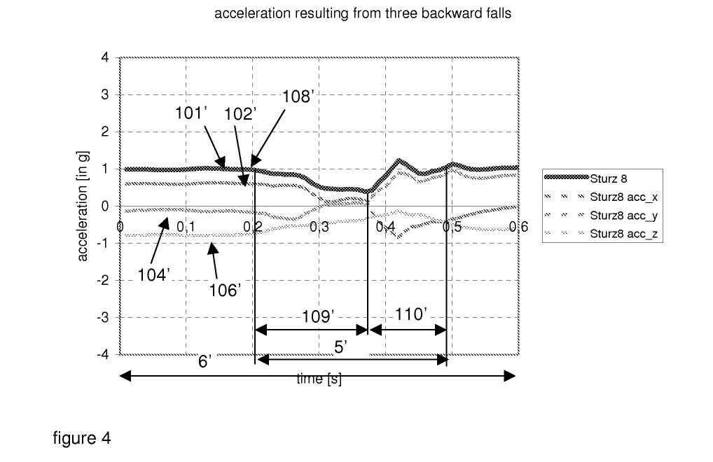

FIGS. 3 and 4 show a diagram of the change of acceleration values over time, said change taking place when a person falls backwards.

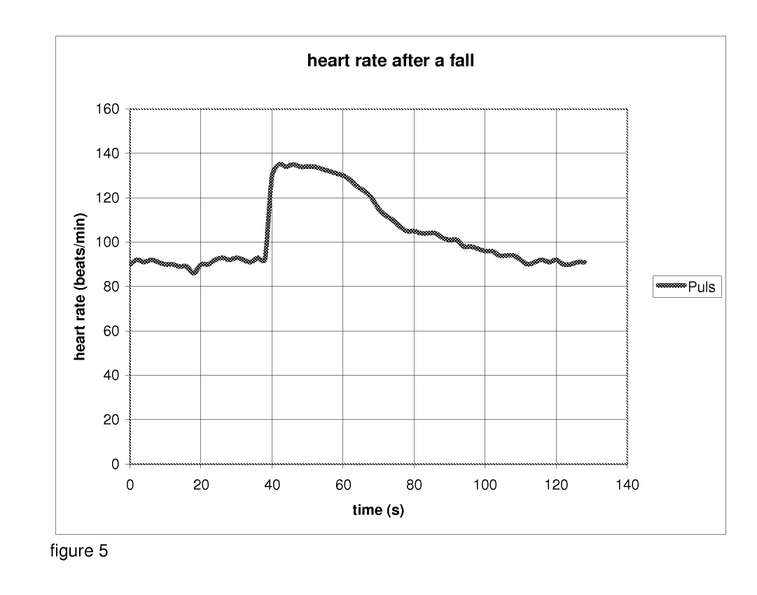

FIG. 5 shows a diagram of the change of the heart rate of a person over a period of time during which a fall occurs.

FIG. 1 shows a flow diagram of the detection of a situation 5 which is abnormal for a person 1, a contact measuring device, such as a contact sensor and/or a pressure sensor and/or a photosensor, and/or a distance measuring device KS 2 being used to determine a contact state and/or the pressure exerted by a body part, such as a foot 3 of a person 1, on a reference surface 4, such as a floor, so that an abnormal situation 5 may be detected among a plurality of different situations 6. The data relating to the contact state and/or the pressure are transmitted to a data collection system DES 201.

The relevant physical data are measured by means of a measuring device for the physiological index PI 7, such as a pulse measuring device, a body conductivity measuring device, etc, and the relevant motion data of the person 1 are measured by means of an activity measuring device AI 8, such as an acceleration or velocity measuring device. These data are then transmitted to a data collection system DES 201.

If necessary, the measured values which are determined by a contact measuring device 2, a measuring device 8 for determining the activity index and a measuring device 7 for determining the physiological index may be radio-transmitted to a central computing and alarm module ZRM 202. The central computing and alarm module 202 is configured to request data concerning contact state or pressure, the physiolocgical index and/or the activity index from the data collection system DES 201, if need be. The data of a certain period of time are analyzed and compared by methods known to those of the art. The analysis of the data is based on individual data or a combination of data.

If an abnormal situation (Case 1, 202) is detected based on individual data or a combination of the acquired data, the rescue forces will be induced to make a monitoring call. If this call of the rescue forces is not answered, an emergency call is made to emergency medical services 297. If the person 1 answers the call, the rescue forces will only intervene after a consultation 208.

If no abnormal situation 204 is detected, no action 209 will be taken.

FIG. 2 shows an example of how the devices required for implementing the method of the invention are arranged on a person 1. A contact sensor and/or a pressure sensor is/are attached to the foot 3 or a shoe in order to determine the contact status of a foot of a person 1 in relation to a reference surface 4, such as a floor. A measuring device 7 for determining the physiological index, such as a heart rate measuring device or a thermometer, is disposed in a position on a body part where the respective physiological data can be measured. The device further comprises at least one measuring device 8 for determining the activity index, such as an acceleration sensor, an inclination sensor, or a velocity measuring device.

The device preferably further comprises an alarm device 9 generating a signal when an abnormal situation is detected and/or the occurrence of an abnormal situation is to be expected, the generation of the signal being controllable by the person 1 or a third person in order to avoid false alarms.

In addition to the contact sensor, the measuring device 7 for determining the physiological index, the measuring device 8 for determining the activity index as well as the device 9 for generating signals, the device for implementing the method of the invention comprises a device 10 for collecting and evaluating data and a position measuring device 11. The above device is arranged in functional units, the first unit comprising at least one of the devices, the second unit comprising the same or at least one other device.

The device or at least one of its units is preferably integrated into glasses 12, a hearing aid 13 or a watch 14 worn by the person. It is preferred to integrate devices for determining the body conductivity (perspiration) into a hearing aid 13 or an ear piece of the glasses 12, while the device for measuring the heart rate is integrated into the watch 14 worn around the wrist.

All the units of the device are configured in a way that measured values are only collected and/or transmitted from the first unit to the second unit, if, based on at least one measured value, the occurrence of an abnormal situation is considered to be possible and/or established. Moreover, the device comprises a position monitoring device in order to make the device 9 for generating signals emit a signal if the device or a unit of the device is removed from the person and/or the communication between the first unit and the second unit is interrupted.

FIGS. 3 and 4 show the change of acceleration figures during a fall 108′ and 108″ over time. The diagrams 101′ and 101″ show the change of the respective acceleration figures resulting from two falls over time. The acceleration values are laid off as ordinate, while the time is laid off as abscissa of the diagram. The diagrams 102′ and 102″ show the acceleration in the x-direction 103, the diagrams 104′ and 104″ show the acceleration in the y-direction 105, the diagrams 106′ and 106″ show the acceleration in the z-direction 107. The falls occur approximately at the instants 108′ and 108″. The occurrence of the falls, which corresponds to the point in time when the abnormal situations which are to be detected and extend over the periods 5′, 5″ set in, is characterized by an initial decrease of the acceleration. During the periods 109′ and 109″, the person falls. The fall or the falling down of the person starts at a point in time at which the acceleration in the z-direction is lower than 1.0 g. The period 110′, 110″ during which the falling person 1 hits a reference area 4, such as a floor, can be identified in FIG. 2 based on the high acceleration figures obtained during this period of time.

The diagrams 101′ and 101″ may be evaluated in relation to the contact state determined by the contact sensor. Comparing the diagrams 101′ and 101″, it can be shown that characteristics of the diagram, such as higher acceleration figures during the period 110′ when the person hits the floor, do not necessarily become apparent. For this reason, it is reasonable to detect abnormal situations based on several data and measured values, in order to avoid misinterpretations.

It is also possible to evaluate the activity values, such as acceleration, motion, position, etc, of a first body part of the person in comparison to the measured values, such as acceleration, motion, position, etc, of a second body part of the person.

FIG. 4 shows the typical shape of a heart rate curve of a person over time before and after the occurrence of a fall, the heart rate being laid off as ordinate, the time being laid off as abscissa. The abnormal event 5 starts with the fall 108 which leads to a sudden increase of the heart rate. The increased heart rate remains high while the person is falling down and hits the floor, and only returns to its normal level after the end of the abnormal event.

1. A method for detecting an abnormal situation of a person, characterized in that a contact measuring device which the person wears directly or indirectly on the body and which is a contact sensor and/or a pressure sensor and/or a photosensor and/or a distance measuring device determine(s) a contact state and/or the pressure exerted by a body part of the person onto a reference surface, so that an abnormal situation can be detected among a plurality of situations based on a change of the contact state and/or of the pressure exerted by the body part of a person onto a reference surface.

2. The method according to claim 1, characterized in that a physiological index of the person is determined and related to the contact state and/or the pressure value.

3. The method according to claim 1, characterized in that an activity index of the person is determined and related to the contact state and/or the pressure value.

4. The method according to claim 1, characterized in that a signal is generated when an abnormal situation is detected or to be expected.

5. The method according to claim 4, characterized in that the generation of the signal is controlled by the person or another person.

6. The method according to claim 2, characterized in that the physiological index is determined based on physiological values, which are preferably averaged over a certain period of time and/or evaluated by means of an analysis method.

7. The method according to claim 2, characterized in that the physiological index is determined based on physiological values of a first body part in relation to the measured values of a second body part, said measured values being preferably averaged over a certain period of time and/or evaluated by means of an analysis method.

8. The method according to claim 3, characterized in that the activity index of the person is determined based on body activity values, such as acceleration, motion, position, etc, said values being preferably averaged over a certain period of time and/or evaluated by means of an analysis method.

9. The method according to claim 3, characterized in that the activity index of the person is determined based on activity values, such as acceleration, motion, position, etc, of a first body part in relation to the measured values, said values being preferably averaged over a certain period of time and/or evaluated by means of an analysis method.

10. A device for implementing the method according to claim 1, said device comprising a contact measuring device, such as a contact sensor and/or a pressure sensor and/or a photosensor, and/or a distance measuring device for determining a contact state or a pressure which is exerted by a body part on a reference surface, said device further comprising one of the following other devices:
a measuring device for determining the physiological index,
a measuring device for determining the activity index,
a device for generating signals,
a device for evaluating data,
a position measuring device.

11. The device according to claim 10, characterized in that the device consists of a first unit and optionally of a second unit, the first unit comprising at least one of the devices and the second device optionally comprising the same or at least one other device, the first unit and the second unit being in communication with each other.

12. The device according to claim 10, characterized in that the device comprises a position monitoring device, so that a signal is generated when the device or a unit of the device is removed from the person and/or the communication between the first unit and the second unit is interrupted.

13. The device according to claim 10, characterized in that the device or at least a unit of a device which is attached to the person is integrated.

14. The device according to claim 10, characterized in that the device is configured in a way that second and optionally further measured values are only collected and/or transmitted from the first unit to the second unit, if, based on at least one measured value, the occurrence of an abnormal situation is considered to be possible and/or established.
